(19) United States
(12) Patent Application Publication
(10) Pub. No.: US 2006/0015110 A1
(43) Pub. Date: Jan. 19, 2006

(54) Cutting device

(76) Inventor: John R. Pepper, Cheshire, CT (US)

Correspondence Address: Charles N.J. Ruggiero, Esq.;Ohlandt, Greeley, Ruggiero & Perle, L.L.P., 10th Floor, One Landmark Square, Stamford, CT, 06901-2682, US

(21) Appl. No.: 10/892,424

(22) Filed: Jul. 15, 2004

(51) Int. Cl. A61B 17/00

(52) U.S. Cl. 606/80

(57) ABSTRACT

A cutter for reaming of the bone canal is provided. The cutter facilitates connection with a guide rod by having a larger target for insertion of the guide rod into the cutter channel of the cutter. The cutter can have an opening with a first portion that is substantially orthogonal to the longitudinal axis of the cutter channel and a second portion that is non-orthogonal to the longitudinal axis. The first and second portions can be orthogonal to each other.

### BACKGROUND OF THE INVENTION

1. Field of the Invention

The present invention relates to bone canal reaming devices and methods. More particularly, the present invention relates to a cutting or reaming device usable with a guide rod.

2. Description of Related Art

The treatment of long bone fractures typically involves internal stabilization. An intramedullary nail or rod, which is typically a cylindrical hollow rod, is inserted in the center of the intramedullary canal or marrow cavity. The rod, which is typically titanium alloy or stainless steel, is strong enough to support the bone loads during the bones healing process. The bones that are fixed in this manner are the femur, tibia, humerus, radius and ulna. Nails are typically generally circular in cross section or have a shape nearly circular.

One step in the surgical technique is the preparation of the canal. The bone canal varies in shape depending upon the position along the bone axis. The center of the bone is called the isthmus, which is a narrowing of the canal. This is especially true in the femur where in order to carry the body weight, the bone gets quite thick in its center.

A nail must be sized to carry the body weight. In order to get present day nails large enough to carry these loads, typically one and a half to three times body weight, the rod must be larger than the canal. What is presently done is enlarging of the canal. To do so, a guide rod is inserted in the canal along its entire length. It is usually about two to about four millimeters in diameter and is about 250 to about 1000 millimeters long. It serves several purposes including aligning the fragments of bone where the surgeon uses the guide rod to thread the segments through their canals, as well as being used to guide cutters through the canal to enlarge it to accept the nail.

The reaming process includes several components. A drill with a hole through its driving axis is used to supply power. Bone is difficult to cut, especially in young males, who are frequently the fracture patient. The drill is either pneumatic or battery powered. It can impart a high torque on all driving components in order to ream the bone. As the canal of the bone is curved, a flexible shaft is used to connect the drill and the reamer. The flexible shaft is usually about 450 millimeters long and about eight to about twelve millimeters in diameter. The flexible shaft is cannulated, and has a hole along its length slightly larger than the guide rod. The flexible shaft has a connector or connection means on one end for the drill and on the other end for connection with the reamer head (cutters).

The reaming set is a number of cutters that increase in diameter in increments of one half to one millimeter. A set of reamer heads for the femur may have reamer heads from nine to fourteen millimeters. The reamers are typical less than three centimeters in length, as longer cutter could not follow the curve of the bone. The reaming is done to one-half to one millimeter over the selected nail size so it is easy to insert.

The reaming is done by attaching a reamer head to the flexible shaft and then threading these two parts over the end of the guide rod. They are advanced to bone, cut the bone and are withdrawn. The cutter is pulled back off the guide rod and then disconnected from the flexible shaft by moving the cutter radially. The next size reamer head is connected to the flexible shaft radially, and then the assembly is axially threaded onto the guide rod. The process is repeated until the desired cavity size is prepared.

It is common for six to ten reaming steps to be needed to make the bone canal of sufficient size to accept a nail. This is a slow and tedious part of the surgery. The difficulty arises in that the guide rod is very long and is only slightly more rigid than a coat hanger. The flexible shaft is also pretty flimsy and almost as long. The guide rod's long length outside the body makes it a hard target for connection with the cutter and the flexible shaft. The guide rod, which is typically three millimeters in diameter, must be axially aligned with a hole in the flexible shaft having a three millimeter nominal size, with only about a quarter millimeter of tolerance. This connection step requires the surgeon to perfectly axially align two long, flexible parts in order to be threaded.

The connection requires the surgeon to hold the dovetail style cutter to the flexible shaft while trying to do the threading process. At the same time, the drill must be supported. This requires at least three hands: one to hold the drill, one to hold the flexible shaft reamer connection and one to hold the guide rod steady so they can be axially aligned. This connection is further complicated because the surgeon's gloves are wet with blood and fat from the canal, rendering them very slick.

The contemporary guide rods are constructed of non-resilient or non-flexible material. This provides additional difficulty in manipulating the cutter or reamer heads onto the guide rod by requiring the drill and drill head to be lifted high into the air to perform the connection or exchange.

Some surgeons that are very skilled can hold the drill with one hand, and use the other to hold the reamer to the shaft, and capture the bouncing guide rod, then align it all together with one hand. Few indeed are those who have this type of dexterity. This is further complicated by the fact that if the reamer head is dropped from the hand onto the floor during this process, it must be sterilized delaying the surgical procedure even further. Clearly, reaming is a frustrating part of the long bone fracture fixation procedure, and it is no wonder it is left to residents and those in their medical training to do this tedious task.

Accordingly, there is a need for a reaming device and related apparatus that addresses these drawbacks.

### SUMMARY OF THE INVENTION

It is an object of the present invention to facilitate the exchanging of cutters during a bone reaming process.

It is another object of the present invention to facilitate the more rapid exchanging of cutters during the bone reaming process.

It is a further object of the present invention to provide a cutter that facilitates the connection of the cutter to a guide rod.

It is a yet another object of the present invention to provide a cutter that facilitates the more rapid connection of the cutter to the guide rod.

It is still another object of the present invention to provide a cutter that allows for a non-axial insertion of the guide rod into the cutter.

These and other objects and advantages of the present invention are provided by a cutter for use with a guide rod for reaming of a bone canal. The cutter has a body with a cutting surface, a distal end, a proximal end and a channel between the distal and proximal ends. The channel has a longitudinal axis. The distal end has an opening in communication with the channel. At least a portion of the opening is disposed in a first plane that is non-orthogonal to the longitudinal axis.

In another embodiment, a cutter for use with a guide rod for reaming of a bone canal is provided that has a body having a cutting surface, a distal end, a proximal end and a channel between the distal and proximal ends. The channel has a cross-sectional area. The distal end has an opening in communication with the channel. The opening has an area that is larger than the cross-sectional area.

In still another embodiment, a reaming device for reaming of a bone canal is provided. The reaming device has a driving device, a guide rod and a cutter. The cutter has a body with a cutting surface, a distal end, a proximal end and a channel between the distal and proximal ends. The channel has a cross-sectional area. The distal end has an opening in communication with the channel, and the opening has an area that is larger than the cross-sectional area.

In a further embodiment, a method of connecting a cutter to a guide rod for reaming of a bone canal is provided which includes, but is not limited to:

(I) providing a guide rod;

(II) providing a cutter that is removably connectable to the guide rod, wherein the cutter has a longitudinal axis, a channel disposed therethrough and an opening in communication with the channel;

(III) loading the cutter on the guide rod by inserting an end of the guide rod into the opening at an angle to the longitudinal axis; and

(IV) advancing said cutter along said guide rod.

In still another embodiment, a method of connecting a cutter to a guide rod for reaming of a bone canal is provided which includes, but is not limited to:

(I) providing a guide rod;

(II) providing a cutter that is removably connectable to the guide rod, wherein the cutter has a longitudinal axis, a channel disposed therethrough and an opening in communication with the channel, and wherein the opening has a radial slot extending along only a portion of the cutter or part way along its length;

(III) loading the cutter on the guide rod by inserting an end of the guide rod through the radial slot; and

(IV) advancing the cutter along the guide rod.

The opening can have first and second portions. The first portion may be in a second plane that is orthogonal to the longitudinal axis and the second portion can be in the first plane. The first plane may be parallel to the longitudinal axis. The opening can be defined at least in part by walls that are splayed. The channel has a longitudinal axis, and the opening may have at least a portion that is in a first plane that is non-orthogonal to the longitudinal axis.

The opening can be defined at least in part by walls that converge in a direction of the longitudinal axis. The opening may also be defined at least in part by walls that diverge in a direction of the distal end. The opening can be defined at least in part by walls that have a chamfered edge.

The connection method may provide for rotating the guide rod toward the longitudinal axis prior to advancing the cutter along the guide rod. The connection method can also provide for inserting the guide rod through the radial slot until it abuts a channel wall that is opposite to the radial slot. The connection method may also provide for locking the cutter to a driving device.

Other and further objects, advantages and features of the present invention will be understood by reference to the following:

### BRIEF DESCRIPTION OF THE DRAWINGS

FIG. 1 is a perspective view of a prior art cutter;

FIG. 2 is plan view of a prior art cutter connected with a flexible shaft;

FIG. 3 is a plan view of the insertion of a guide rod into the prior art cutter of FIG. 1 for connection;

FIG. 4 is a plan view of the prior art cutter being advanced along the guide rod with the flexible shaft not shown;

FIG. 5 is a perspective view of a cutter of the present invention;

FIG. 6 is a plan view of the cutter of FIG. 5;

FIG. 7 is a perspective cross sectional view of the cutter of FIG. 6 taken along line 7-7 of FIG. 6;

FIG. 8 is a top view of the cutter of FIG. 5;

FIG. 9 is a perspective view of the cutter of FIG. 5 connected to a flexible shaft;

FIG. 10 is a perspective view of a guide rod partially inserted into the cutter of FIG. 5;

FIG. 11 is a cross-sectional view of a first method of inserting a guide rod into the cutter of FIG. 5;

FIG. 12 is a cross-sectional view of a second method of inserting the guide rod into the cutter of FIG. 5;

FIG. 13 is a cross-sectional view of an alternative embodiment of a cutter of the present invention;

FIG. 14 is an enlarged view of the upper portion of the cutter of FIG. 13 including the opening;

FIG. 15 is a perspective view of another alternative embodiment of a cutter of the present invention;

FIG. 16 is a perspective view of another alternative embodiment of a cutter of the present invention; and

FIG. 17 is a perspective view of another alternative embodiment of a cutter of the present invention.

### DETAILED DESCRIPTION OF THE INVENTION

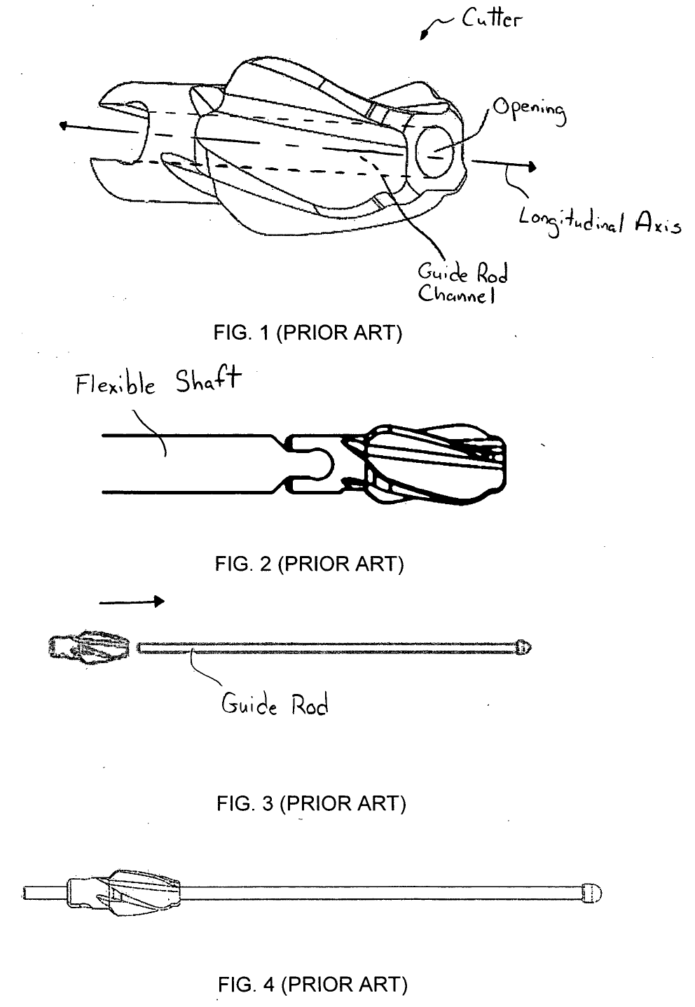

Referring to the drawings, and in particular FIGS. 1 through 4, a prior art cutter is shown, as well as the prior art method of connecting or loading the cutter with a guide rod. A guide rod bore or channel of the prior art cutter is along the longitudinal axis of the cutter and defines an opening that is perpendicular to the longitudinal axis.

In order to connect the prior art cutter with the guide rod, the surgeon must align the guide rod along the longitudinal axis of the cutter and insert it through the opening. The cutter then travels along the guide rod, which is disposed through the guide rod channel of the cutter. During the surgical procedure, this is a difficult task at best, especially due to the tight tolerance between the central bore in the reamer and the outer surface of the guide rod.

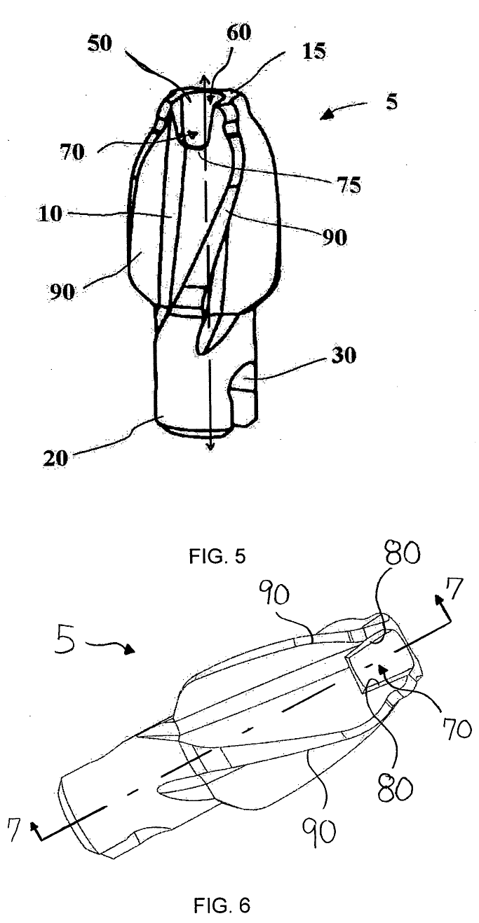

Referring to FIGS. 5 through 10, a cutter of the present invention is shown and generally represented by reference numeral 5. Cutter 5 has a body 10 with a distal end 15, a proximal end 20 and a guide rod bore or channel 25 disposed therebetween. The channel 25 is centrally located along the longitudinal axis of the cutter 5 to define the center bore of the cutter. Preferably, the channel 25 is cylindrical or substantially cylindrical in shape, with a uniform diameter.

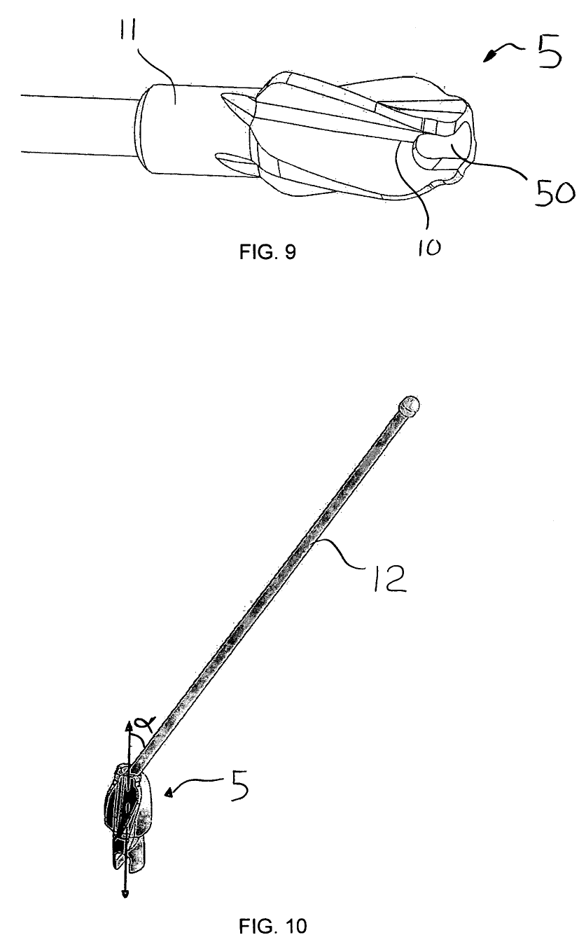

Proximal end 20 of the cutter 5 has a shaft connector 30. The shaft connector 30 provides for connection of a flexible shaft 11 (shown in FIG. 9) with the cutter 5. The shaft connector 30 can have various connection structures, methods or means, which provide for releasable connection of the flexible shaft 11 and the cutter 5. Additionally, the present invention contemplates the use of cutter 5 with a shaft 11 that is integrally fixed thereto.

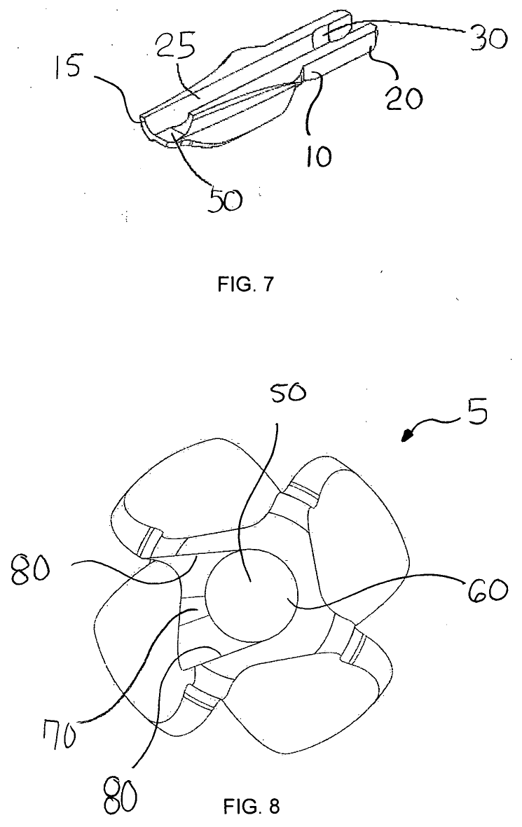

The cutter 5 has an opening 50 disposed therethrough at the distal end 15 of the cutter. In the preferred embodiment of cutter 5, opening 50 is defined by a first portion 60 that is substantially perpendicular or orthogonal to the longitudinal axis of the cutter 5 and a second portion or radial slot 70 that is substantially non-perpendicular or non-orthogonal to the longitudinal axis of the cutter, as shown in FIG. 8. Preferably, the first portion 60 of opening 50 lies in a plane that is perpendicular to the longitudinal axis of the cutter 5 and the second portion 70 of the opening lies in a plane that is parallel to the longitudinal axis. However, the present invention contemplates the opening having portions that are at different angles with respect to each other and to the longitudinal axis of the cutter channel 25, as shown in FIG. 7.

The second portion 70 of the opening 50 is preferably defined by a rim 75 having a substantially U-like shape, as shown in FIG. 5. The size and shape of first portion 60 of the opening 50 is adaptable for receiving the guide rod 12 (shown in FIG. 10) and allowing it to slide therethrough into position. The size and shape of the second portion 70 is adaptable for facilitating the insertion of the guide rod 12 into the opening 50 and allowing it to be moved into axial alignment with the first portion 60 of the opening and the longitudinal axis of the cutter 5. Second portion 70 also has a size and shape that maintains the strength and integrity of the cutter 5 and, in particular, the distal end 15 of the cutter.

The second portion 70 of the opening 50 preferably has a width that is equal to or nearly equal to the cutter channel 25. The height of the second portion 70 of the opening 50 allows for insertion of the guide rod 12 therethrough, while preventing a large stress riser on the cutter 5 or creating an opening for bone fragments to become lodged in.

The rim 75 of the second portion 70 of opening 50 is preferably defined or partially defined by non-parallel walls 80, such as, for example, splayed walls, as shown in FIG. 6. The walls 80 in this preferred embodiment converge toward the longitudinal axis and diverge in the direction of the distal end 15 of cutter 5 (as shown in FIGS. 6 and 8). The non-parallel alignment or configuration of the walls 80 with respect to each other further facilitates the insertion of the guide rod 12 into the second portion 70 of the opening 50 by increasing the target that is presented by the second portion. However, the present invention contemplates the use of other configurations of the walls 80, such as, for example, parallel, partially parallel, and/or partially splayed, where these configurations facilitate insertion of the guide rod 12 into the guide rod channel 25 of the cutter 5.

The body 10 of cutter 5 has a number of cutting flutes 90, as shown in FIG. 5. The opening 50 is preferably positioned between a pair of the cutting flutes 90. This allows for easier access to the opening 50, while also maintaining the cutting efficiency of the cutter 5 by not removing any of the cutting surfaces of the cutting flutes 90. While the preferred embodiment uses a number of cutting flutes 90, the present invention contemplates the use of other structures, methods or means for cutting or reaming of the bone canal, such as alternative cutting surfaces. Additionally, the present invention contemplates opening 50 being positioned over a portion of one or more of the cutting flutes 90, which would remove a portion of the cutting surface. Further, the cutting surfaces preferably are a number of cutting surfaces (for one or more cutting flutes 90), and more preferably are between three and eight cutting edges.

As shown in FIG. 10, the opening 50 having first and second portions 60 and 70 (shown in FIG. 5) allows the guide rod 12 to be initially inserted into the opening at an insertion angle a with respect to the longitudinal axis of the cutter 5 and channel 25. During surgery, where the guide rod 12 can be extended a great length above the patient, this non-axial insertion greatly facilitates the ability to connect the cutter 5 with the guide rod. In the preferred embodiment of cutter 5, insertion angle a is up to at least 90°.

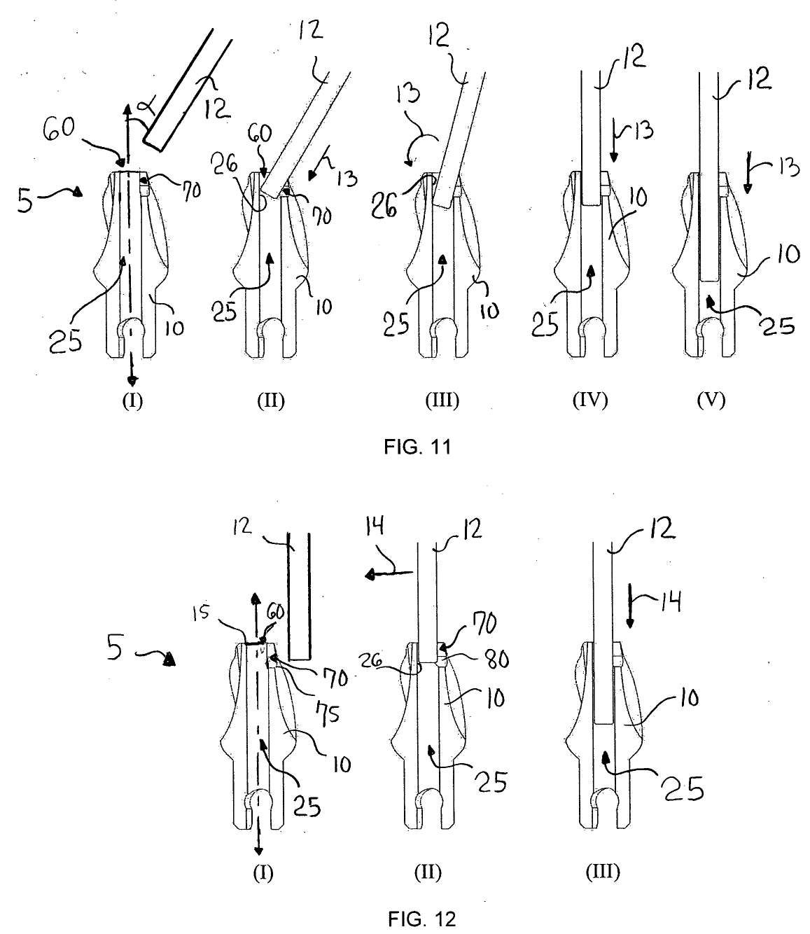

Referring to FIG. 11, a method of connecting the cutter 5 with the guide rod 12 is shown in the steps I through V. The direction of movement of the guide rod 12 with respect to the cutter 5 is shown by arrow 13. The guide rod 12 is initially non-axially aligned with the cutter 5 at an insertion angle α, as shown in step I. The end of the guide rod 12 is then inserted into opening 50 through first and second portions 60 and 70, as shown in step II.

The opening 50 is comprised of a portion (second portion 60) that lies in a plane that is non-orthogonal to the longitudinal axis of the cutter 5, which results in the overall size of the opening being larger than the size or diameter of guide rod channel 25. The size of the portion (first portion 60) of the opening 50 along the distal end 15 of the cutter 5 is equal to or nearly equal to the size or diameter of the channel 25. The overall increased size of opening 50 facilitates connection of the cutter 5 with the guide rod 12 by providing a much larger target for insertion.

A channel wall 26, which is disposed opposite to the second portion 70 of opening 50, acts as a guide for the placement of the end of the guide rod 12 into the guide rod channel 25. Once partially inserted therein, the guide rod 12 can be rotated into axial alignment with the cutter 5 and channel 25 using channel wall 26 as an abutment or guide, as shown in steps III and IV. The channel wall 26 and rim 75 of opening 50 acts as a hinge for the rotation of the guide 12 rod into axial alignment with the cutter 5. The guide rod 12 then travels along the channel 25, as shown in step V.

Referring to FIG. 12, an alternative method of connecting the cutter 5 with the guide rod 12 is shown in the steps I through III. The direction of movement of the guide rod 12 with respect to the cutter 5 is shown by arrow 14. The guide rod 12 is initially aligned parallel or substantially parallel with the longitudinal axis of the cutter 5 and channel 25, as shown in step 1. The end of the guide rod 12 is below the distal end 15 of the cutter 5 but above the lower most portion of the rim 75 of opening 50.

The end of the guide rod 12 is then moved towards the cutter 5 and passes through the second portion 70 of opening 50, as shown in step II. This movement is facilitated by the splayed walls 80, which create a larger opening than the diameter of the guide rod 12. The channel wall 26 acts as a stopper for the placement of the end of the guide rod 12 in the channel 25. Once partially inserted therein, the guide rod 12 travels along the channel 25, as shown in step III.

Additionally, a combination of the insertion methods described above with respect to FIGS. 11 and 12 can be utilized with the present invention. The guide rod 12 can be aligned at the angle α and also below the distal end 15 but above the lower most portion of the rim 75 of opening 50. The end of the guide rod 12 is moved through the second portion 70 of opening 50. The guide rod 12 is then also rotated into axial alignment with the longitudinal axis of the cutter 5 and the channel 25, and travels along the cutter channel into position. The channel wall 26 acts as both a stopper and a hinge to facilitate the insertion of the guide rod 12 through the guide rod channel 25 of cutter 5.

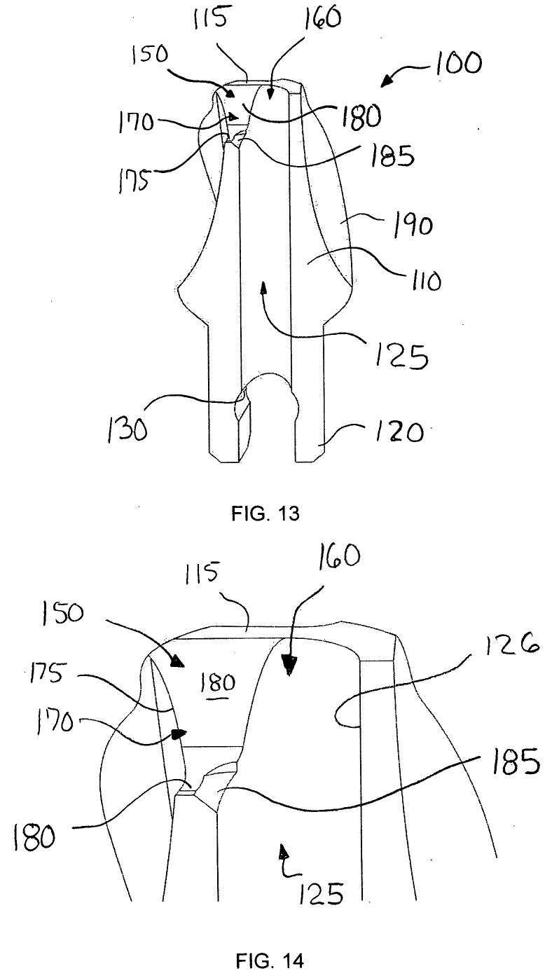

Referring to FIG. 13, an alternative embodiment of the cutter of the present invention is shown and generally represented by reference numeral 100. The cutter 100 has features similar to the features of cutter 5 such as a body 110, a distal end 115, a proximal end 120, a guide rod bore or channel 125 disposed therebetween, a shaft connector 130, an opening 150 disposed through the distal end that has first and second portions 160 and 170, and cutting flutes 190.

The opening 150 is defined by a rim 175 that is formed in part by splayed wall 180. A lower portion of the wall 180 has a chamfered, angled or non-symmetrical edge 185. The chamfered edge 185 further facilitates the insertion of the guide rod 12 into the guide rod channel 125 by eliminating and/or smoothing a portion of the edge. The guide rod 12 is rotated into axial alignment with the cutter 100 and the channel 25 using both a channel wall 126 and the chamfered edge 185 as a hinge or guide. Additionally, the chamfered edge 185 facilitates sliding of the guide rod 12 along the channel 125 since the edge has been eliminated and/or smoothed over reducing friction.

The present invention has first and second portions 60 or 160 and 70 or 170, which increase the area or size of opening 50 or 150, respectively. These portions are orthogonal or substantially orthogonal to each other. However, the present invention contemplates the use of other angles, sizes or shapes for the openings 50 or 150 in order to facilitate the insertion of the guide rod 12 into the cutter 5 or 100. The present invention also contemplates the use of other features on the cutters 5 or 100, such as, for example, alternative types of cutting flutes 90 or 190, as well as a variety of materials or combinations of materials from which the cutters 5 or 100 can be made.

The present invention contemplates the use of more than two portions for openings 50 or 150 that are positioned at different angles with respect to each other and/or the longitudinal axis of the guide rod channel 25 or 125 of the cutters 5 and 100, respectively.

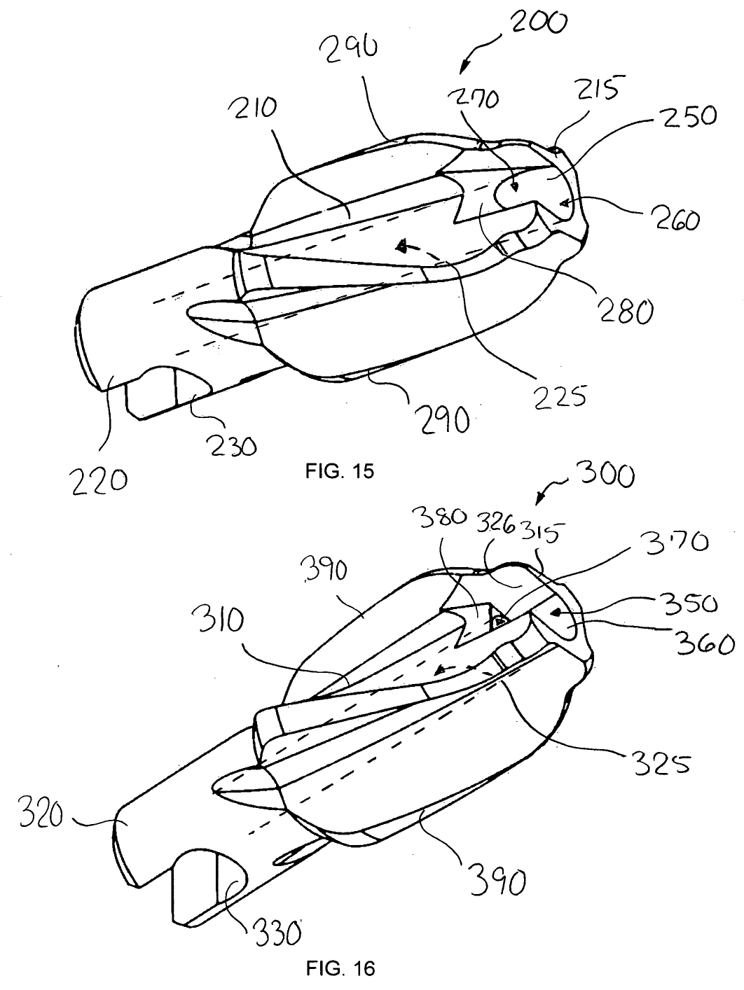

Referring to FIG. 15, another alternative embodiment of the cutter of the present invention is shown and generally represented by reference numeral 200. The cutter 200 has features similar to the features of cutter 5 such as a body 210, a distal end 215, a proximal end 220, a guide rod bore or channel 225 disposed therebetween, a shaft connector 230, an opening 250 disposed through the distal end that has first and second portions 260 and 270, and cutting flutes 290.

The opening 150 is defined in part by an angled wall 280. First and second portions 260 and 270 increase the size of opening 250. First and second portions 260 and 270 are non-orthogonal to each other. The angle with respect to the longitudinal axis of the guide rod channel 225 at which the second portion 270 lies, as well as the slope or angle of wall 280 facilitates insertion of the guide rod 12 into the opening 250. This can be especially useful where the guide rod 12 is being inserted into the cutter 200 at a large angle a (shown in FIG. 11), as well as where the end of the guide rod is near or abutting the body 210 so that the end of the guide rod can be slid more easily over and into the opening 250.

Referring to FIG. 16, yet another alternative embodiment of the cutter of the present invention is shown and generally represented by reference numeral 300. The cutter 300 has features similar to the features of cutter 5 such as a body 310, a distal end 315, a proximal end 320, a guide rod bore or channel 325 disposed therebetween, a shaft connector 330, an opening 350 disposed through the distal end that has first and second portions 360 and 370, and cutting flutes 390.

The opening 350 has first and second portions 360 and 370 that are disposed substantially parallel to each other but are offset from each other along the length of cutter 300. First portion 360 has a substantially U-like shape and is offset from second portion 370 by a first wall 326. The first wall 326 is defined partially by the body 310 and the cutting flutes 390. The first wall 326 acts as both a stopper and a hinge to facilitate the insertion of the guide rod 12 through second portion 370 and then the guide rod channel 325 of cutter 300. Second portion 370 is defined in part by a second wall 380. Preferably second wall 380 is chamfered or angled inwardly to facilitate insertion of the guide rod 12 into the opening 350. This can be especially useful where the guide rod 12 is being inserted into the cutter 300 at a large angle a (shown in FIG. 11), as well as where the end of the guide rod is near or abutting the body 310 so that the end of the guide rod can be slid more easily over and into the opening 350.

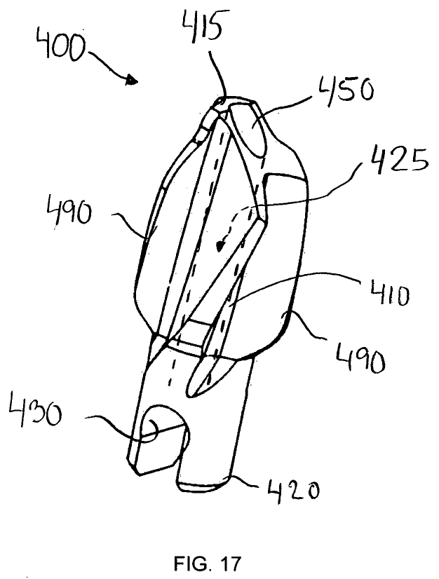

Referring to FIG. 17, still another alternative embodiment of the cutter of the present invention is shown and generally represented by reference numeral 400. The cutter 400 has features similar to the features of cutter 5 such as a body 410, a distal end 415, a proximal end 420, a guide rod bore or channel 425 disposed therebetween, a shaft connector 430, an opening 450 disposed through the distal end, and cutting flutes 390.

Cutter 400 provides for the use of a single opening, e.g., an opening in a single plane or a substantially single plane, where the plane is non-orthogonal to the longitudinal axis of the cutter. This type of angled opening allows for an enlarged area for insertion, as compared to the diameter of the center bore channel, and allows for insertion at an angle to the longitudinal axis of the cutter.

The present invention having been thus described with particular reference to the preferred forms thereof, it will be obvious that various changes and modifications may be made therein without departing from the spirit and scope of the present invention as defined in the appended claims.

What is claimed is:

1. A cutter for use with a guide rod for reaming of a bone canal, the cutter comprising:
a body having at least one cutting surface, a distal end, a proximal end and a channel between said distal and proximal ends, said channel having a longitudinal axis, wherein said distal end has an opening in communication with said channel, wherein at least a portion of said opening is in a first plane that is non-orthogonal to said longitudinal axis, wherein said at least a portion of said opening extends only partially along said body, and wherein said channel has a size and shape to receive the guide rod.

2. The cutter of claim 1, wherein said opening has first and second portions, wherein said first portion is in a second plane that is orthogonal to said longitudinal axis and said second portion is in said first plane.

3. The cutter of claim 2, wherein said first plane is parallel to said longitudinal axis.

4. The cutter of claim 2, wherein said opening is defined at least in part by a plurality of walls that are splayed.

5. The cutter of claim 4, wherein said plurality of walls converge in a direction of said longitudinal axis.

6. The cutter of claim 4, wherein said plurality of walls diverge in a direction of said distal end.

7. The cutter of claim 6, wherein said plurality of walls have a chamfered edge.

8. A cutter for use with a guide rod for reaming of a bone canal, the cutter comprising:
a body having at least one cutting surface, a distal end, a proximal end and a channel between said distal and proximal ends, said channel having a cross-sectional area, wherein said distal end has an opening in communication with said channel, wherein said opening has an area that is larger than said cross-sectional area, wherein said opening extends only partially along said body, and wherein said channel has a size and shape to receive the guide rod.

9. The cutter of claim 8, wherein said channel has a longitudinal axis, and wherein said opening has at least a portion that is in a first plane that is non-orthogonal to said longitudinal axis.

10. The cutter of claim 9, wherein said opening has first and second portions, wherein said first portion is in a second plane that is orthogonal to said longitudinal axis and said second portion is in said first plane.

11. The cutter of claim 10, wherein said first plane is parallel to said longitudinal axis.

12. The cutter of claim 9, wherein said opening is defined at least in part by a plurality of walls that converge in a direction of said longitudinal axis.

13. The cutter of claim 9, wherein said opening is defined at least in part by a plurality of walls that diverge in a direction of said distal end.

14. The cutter of claim 9, wherein said opening is defined at least in part by a plurality of walls that have a chamfered edge.

15. A reaming device for reaming of a bone canal comprising:
a driving device;
a guide rod; and
a cutter, wherein said cutter has a body with at least one cutting surface, a distal end, a proximal end and a channel between said distal and proximal ends, wherein said channel has a cross-sectional area, wherein said distal end has an opening in communication with said channel, wherein said opening has an area that is larger than said cross-sectional area, wherein said opening extends only partially along said body, and wherein said channel has a size and shape to receive said guide rod.

16. The reaming device of claim 15, wherein said channel has a longitudinal axis, and wherein said opening has at least a portion that is in a first plane that is non-orthogonal to said longitudinal axis.

17. The reaming device of claim 16, wherein said opening has first and second portions, wherein said first portion is in a second plane that is orthogonal to said longitudinal axis and said second portion is in said first plane.

18. The reaming device of claim 17, wherein said first plane is parallel to said longitudinal axis.

19. The reaming device of claim 16, wherein said opening is defined at least in part by a plurality of walls that converge in a direction of said longitudinal axis.

20. The reaming device of claim 16, wherein said opening is defined at least in part by a plurality of walls that diverge in a direction of said distal end.

21. The reaming device of claim 16, wherein said opening is defined at least in part by a plurality of walls having at least a portion with a chamfered edge.

22. A method of connecting a cutter to a guide rod for reaming of a bone canal, the method comprising:
providing a guide rod;
providing a cutter that is removably connectable to said guide rod, said cutter having a longitudinal axis, a channel disposed therethrough and an opening in communication with said channel, said channel having a size and shape to receive said guide rod, said opening extending only partially along said cutter;
loading said cutter on said guide rod by inserting an end of said guide rod into said opening at an angle to said longitudinal axis; and
advancing said cutter along said guide rod.

23. The method of claim 22, further comprising rotating said guide rod toward said longitudinal axis prior to advancing said cutter along said guide rod.

24. The method of claim 22, further comprising locking said cutter to a driving device.

25. A method of connecting a cutter to a guide rod for reaming of a bone canal, the method comprising:
providing a guide rod;
providing a cutter that is removably connectable to said guide rod, said cutter having a longitudinal axis, a channel disposed therethrough and an opening in communication with said channel, said channel having a size and shape to receive said guide rod, said opening having a radial slot extending only partially along said cutter;
loading said cutter on said guide rod by inserting an end of said guide rod through said radial slot; and
advancing said cutter along said guide rod.

26. The method of claim 25, wherein said channel has a channel wall opposite to said radial slot, and wherein said end of said guide rod is inserted through said radial slot until abutting said channel wall.

27. The method of claim 25, further comprising locking said cutter to a driving device.
